(19) United States
(12) Patent Application Publication
(10) Pub. No.: US 2014/0336196 A1
(43) Pub. Date: Nov. 13, 2014

(54) PHOSPHORIC ACID SALTS OF SITAGLIPTIN

(71) Applicant: Merck Sharp & Dohme Corp., Rahway, NJ (US)

(72) Inventor: Jerry L. Atwood, Columbia, MO (US)

(73) Assignee: Merck Sharp & Dohme Corp., Rahway, NJ (US)

(21) Appl. No.: 14/122,002

(22) PCT Filed: May 22, 2012
(86) PCT No.: PCT/US2012/038917

§ 371 Date: Nov. 25, 2013

Related U.S. Application Data
(60) Provisional application No. 61/490,819, filed on May 27, 2011.

(51) Int. Cl. C07D 487/04

(52) U.S. Cl. CPC C07D 487/04
USPC 514/249; 544/350

(57) ABSTRACT

The present invention relates to novel phosphoric acid salts of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, and polymorphs, hydrates and solvates thereof, which are potent inhibitors of dipeptidyl peptidase-IV useful for the prevention and/or treatment of non-insulin dependent diabetes mellitus, also referred to as type 2 diabetes. The present invention also relates to the process for preparing the novel phosphoric acid salts of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, as well as pharmaceutical compositions containing the novel phosphoric acid salts, and methods of use thereof for the treatment of diabetes, obesity, and high blood pressure.

### FIELD OF THE INVENTION

The present invention relates to novel phosphoric acid salts of 4-oxo-4-[3-(trifluoromethyl)-5,6-dihydro[1,2,4]-triazolo[4,3-a]pyrazin-7(8H)-yl]-1-(2,4,5-trifluorophenyl)butan-2-amine, also known as sitagliptin, which is a potent inhibitor of dipeptidyl peptidase-IV. More particularly, the invention relates to the bis(sitagliptin) phosphoric acid salt, the sitagliptin ammonia phosphoric acid salt, and the sitagliptin bis(phosphoric acid) salt, and polymorphs, hydrates and solvates thereof. These novel phosphoric acid salts, and their polymorphs, hydrates and solvates, are useful for the treatment and prevention of diseases and conditions for which an inhibitor of dipeptidyl peptidase-IV is indicated, in particular Type 2 diabetes, obesity, and high blood pressure. The invention further concerns pharmaceutical compositions comprising the bis(sitagliptin) phosphoric acid salt, the sitagliptin ammonia phosphoric acid salt, and the sitagliptin bis(phosphoric acid) salt, and polymorphs, hydrates and solvates thereof, and methods of using these novel phosphoric acid salts, and their polymorphs, hydrates and solvates, to treat Type 2 diabetes, obesity, and high blood pressure. The invention further concerns processes for preparing the novel phosphoric acid salts of 4-oxo-4-[3-(trifluoromethyl)-5,6-dihydro[1,2,4]-triazolo[4,3-a]pyrazin-7(8H)-yl]-1-(2,4,5-trifluorophenyl)butan-2-amine, and polymorphs, hydrates and solvates thereof.

### BACKGROUND OF THE INVENTION

Inhibition of dipeptidyl peptidase-IV (DP-IV), an enzyme that inactivates both glucose-dependent insulinotropic peptide (GIP) and glucagon-like peptide 1 (GLP-1), represents a novel approach to the treatment and prevention of Type 2 diabetes, also known as non-insulin dependent diabetes mellitus (NIDDM). The therapeutic potential of DP-IV inhibitors for the treatment of Type 2 diabetes has been reviewed: C. F. Deacon and J. J. Holst, “Dipeptidyl peptidase IV inhibition as an approach to the treatment and prevention of Type 2 diabetes: a historical perspective,” Biochem. Biophys. Res. Commun., 294: 1-4 (2000); K. Augustyns, et al., “Dipeptidyl peptidase IV inhibitors as new therapeutic agents for the treatment of Type 2 diabetes,” Expert. Opin. Ther. Patents, 13: 499-510 (2003); and D. J. Drucker, “Therapeutic potential of dipeptidyl peptidase IV inhibitors for the treatment of Type 2 diabetes,” Expert Opin. Investig. Drugs, 12: 87-100 (2003).

WO 03/004498 (published 16 Jan. 2003), assigned to Merck & Co., describes a class of beta-amino tetrahydrotriazolo[4,3-a]pyrazines, which are potent inhibitors of DP-IV and therefore useful for the treatment of Type 2 diabetes. Specifically disclosed in WO 03/004498 is 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine. Pharmaceutically acceptable salts of this compound are generically encompassed within the scope of WO 03/004498. WO 05/003135, assigned to Merck & Co., describes other phosphoric acid salts of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine.

### SUMMARY OF THE INVENTION

The present invention is concerned with novel phosphoric acid salts of the dipeptidyl peptidase-IV (DP-IV) inhibitor 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine and polymorphs, hydrates and solvates thereof, in particular the bis(sitagliptin) phosphoric acid monohydrate salt, the bis(sitagliptin) monohydrogen phosphate trihydrate salt, the sitagliptin ammonia phosphoric acid 2.5 hydrate salt, and the sitagliptin bis(phosphoric acid) salt. The novel phosphoric acid salts, polymorphs and hydrates of the present invention have advantages in the preparation of pharmaceutical compositions of 4-oxo-4-[3-(trifluoromethyl)-5,6-dihydro[1,2,4]-triazolo[4,3-a]pyrazin-7(8H)-yl]-1-(2,4,5-trifluorophenyl)butan-2-amine, such as physical stability and the resulting ease of processing, handling, and dosing. The invention also concerns pharmaceutical compositions containing the novel phosphoric acid salts, polymorphs, hydrates and solvates thereof, as well as methods for using them as DP-IV inhibitors, in particular for the prevention or treatment of Type 2 diabetes, obesity, and high blood pressure.

### BRIEF DESCRIPTION OF THE FIGURES

FIG. 1 is a X-ray diffraction pattern of the crystalline trihydrate of the bis(sitagliptin) phosphoric acid salt of structural formula III-a.

FIG. 2 is a thermogravimetric analysis (TGA) curve of the crystalline trihydrate of the bis(sitagliptin) phosphoric acid salt of structural formula III-a.

FIG. 3 is a differential scanning calorimetry (DSC) curve of the crystalline trihydrate of the bis(sitagliptin) phosphoric acid salt of structural formula III-a.

FIG. 4 is a X-ray diffraction pattern of the crystalline monohydrate of the bis(sitagliptin) phosphoric acid salt of structural formula IV-a.

FIG. 5 is a thermogravimetric analysis (TGA) curve of the crystalline monohydrate of the bis(sitagliptin) phosphoric acid salt of structural formula IV-a.

FIG. 6 is a differential scanning calorimetry (DSC) curve of the crystalline monohydrate of the bis(sitagliptin) phosphoric acid salt of structural formula IV-a.

FIG. 7 is a X-ray diffraction pattern of the crystalline 2.5 hydrate of the sitagliptin ammonia phosphoric acid salt of structural formula VI-a.

FIG. 8 is a thermogravimetric analysis (TGA) curve of the crystalline 2.5 hydrate of the sitagliptin ammonia phosphoric acid salt of structural formula VI-a.

FIG. 9 is a differential scanning calorimetry (DSC) curve of the crystalline 2.5 hydrate of the sitagliptin ammonia phosphoric acid salt of structural formula VI-a.

FIG. 10 is a X-ray diffraction pattern of the amorphous sitagliptin bis(phosphoric acid) salt of structural formula VII-a.

FIG. 11 is a thermogravimetric analysis (TGA) curve of the amorphous sitagliptin bis(phosphoric acid) salt of structural formula VII-a.

### DETAILED DESCRIPTION OF THE INVENTION

This invention provides novel phosphoric acid salts of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine (sitagliptin), which is the compound of structural formula I:

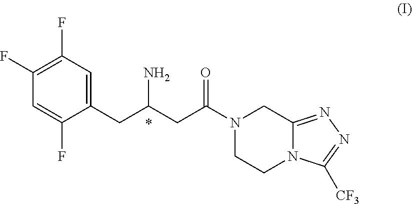 [SMILES: NC(CC(=O)N1CCn2c(nnc2C(F)(F)F)C1)Cc1cc(F)c(F)cc1F]

and polymorphs, hydrates and solvates thereof. In a class of this embodiment, the present invention provides a bis-[4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid trihydrate salt, a bis-[4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid monohydrate salt, a 4-oxo-4-[3-(trifluoromethyl)-5,6-dihydro[1,2,4]triazolo[4,3-a]pyrazin-7(8H)-yl]-1-(2,4,5-trifluorophenyl)-butan-2-amine ammonia phosphoric acid 2.5 hydrate salt, and a 4-oxo-4-[3-(trifluoromethyl)-5,6-dihydro[1,2,4]triazolo[4,3-a]pyrazin-7(8H)-yl]-1-(2,4,5-trifluorophenyl)-butan-2-amine bis(phosphoric acid) salt, and polymorphs, and solvates thereof.

The phosphoric acid salts of the present invention has a center of asymmetry at the stereogenic carbon atom indicated with an * and can thus occur as a racemate, racemic mixture, and single enantiomers, with all isomeric forms being included in the present invention. The separate enantiomers, substantially free of the other, are included within the scope of the invention, as well as mixtures of the two enantiomers.

One embodiment of the present invention provides novel phosphoric acid salts of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, which is the compound of structural formula I-a (also known as sitagliptin free base):

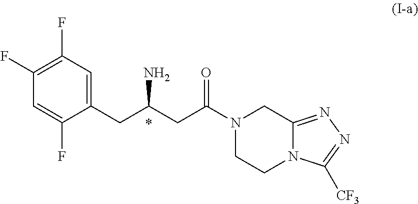 [SMILES: N[C@@H](CC(=O)N1CCn2c(nnc2C(F)(F)F)C1)Cc1cc(F)c(F)cc1F]

and polymorphs, hydrates and solvates thereof. In a class of this embodiment, the present invention provides a bis(sitagliptin) phosphoric acid trihydrate salt, a bis(sitagliptin) phosphoric acid monohydrate salt, a sitagliptin ammonia phosphoric acid 2.5 hydrate salt, and a sitagliptin bis(phosphoric acid) salt, and polymorphs, hydrates and solvates thereof.

Another embodiment of the present invention provides novel phosphoric acid salts of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, which is the compound of structural formula I-b:

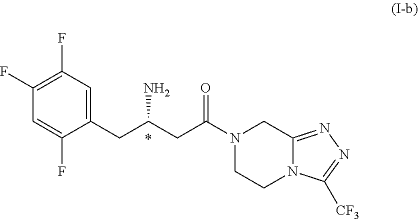 [SMILES: N[C@H](CC(=O)N1CCn2c(nnc2C(F)(F)F)C1)Cc1cc(F)c(F)cc1F]

and polymorphs, hydrates and solvates thereof. In a class of this embodiment, the present invention provides a bis-[(2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid trihydrate salt, a bis-[(2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid monohydrate salt, a (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine ammonia phosphoric acid 2.5 hydrate salt, and a (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine bis(phosphoric acid) salt, and polymorphs, and solvates thereof.

Another embodiment of the present invention provides the bis-[4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid salt of structural formula II:

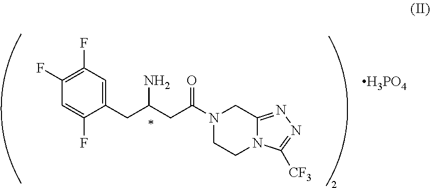

or a polymorph, hydrate and/or solvate thereof. In a class of this embodiment, the salt of structural formula II is a hydrate. In another class of this embodiment, the salt of structural formula II is crystalline. In another class of this embodiment, the salt of structural formula II is a crystalline hydrate. In another class of this embodiment, the salt of structural formula II is a trihydrate. In a subclass of this class, the trihydrate salt of structural formula II is crystalline. In another class of this embodiment, the salt of structural formula II is a monohydrate. In a subclass of this class, the monohydrate salt of structural formula II is crystalline.

The salt of structural formula II is comprised of two molar equivalents of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, and one molar equivalent of phosphoric acid (H₃PO₄).

The trihydrate salt of structural formula II is comprised of two molar equivalents of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of phosphoric acid (H₃PO₄), and three molar equivalents of water.

The monohydrate salt of structural formula II is comprised of two molar equivalents of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of phosphoric acid (H₃PO₄), and one molar equivalent of water.

Another embodiment of the present invention provides the bis(sitagliptin) phosphoric acid salt, which corresponds to the bis-[(2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid salt of structural formula II-a:

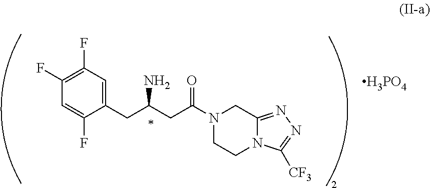

or a polymorph, hydrate and/or solvate thereof. In a class of this embodiment, the salt of formula II-a is a hydrate. In another class of this embodiment, the salt of formula II-a is crystalline. In another class of this embodiment, the salt of formula II-a is a crystalline hydrate. In another class of this embodiment, the salt of formula II-a is a trihydrate. In a subclass of this class, the trihydrate salt of formula II-a is crystalline. In another class of this embodiment, the salt of formula II-a is a monohydrate. In a subclass of this class, the monohydrate salt of formula II-a is crystalline.

The bis(sitagliptin) phosphoric acid salt of the present invention is comprised of two molar equivalents of (2R)-4-oxo-4-[3-(trifluoromethyl)-5,6-dihydro[1,2,4]triazolo-[4,3-a]pyrazin-7(8H)-yl]-1-(2,4,5-trifluorophenyl)butan-2-amine, and one molar equivalent of phosphoric acid (H₃PO₄).

The bis(sitagliptin) phosphoric acid trihydrate salt of the present invention is comprised of two molar equivalents of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of phosphoric acid (H₃PO₄), and three molar equivalents of water.

The bis(sitagliptin) phosphoric acid monohydrate salt of the present invention is comprised of two molar equivalents of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent phosphoric acid (H₃PO₄), and one molar equivalent of water.

Another embodiment of the present invention provides the bis-[(2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid salt of structural formula II-b:

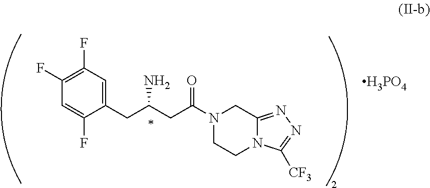

or a polymorph, hydrate and/or solvate thereof. In a class of this embodiment, the salt of structural formula II-b is a hydrate. In another class of this embodiment, the salt of structural formula II-b is crystalline. In another class of this embodiment, the salt of structural formula II-b is a crystalline hydrate. In another class of this embodiment, the salt of structural formula II-b is a trihydrate. In a subclass of this class, the trihydrate salt of structural formula II-b is crystalline. In another class of this embodiment, the salt of structural formula II-b is a monohydrate. In a subclass of this class, the monohydrate salt of structural formula II-b is crystalline.

The salt of structural formula II-b is comprised of two molar equivalents of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, and one molar equivalent of phosphoric acid (H₃PO₄).

The trihydrate salt of structural formula II-b is comprised of two molar equivalents of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of phosphoric acid (H₃PO₄), and three molar equivalents of water.

The monohydrate salt of structural formula II-b is comprised of two molar equivalents of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of phosphoric acid (H₃PO₄), and one molar equivalent of water.

Another embodiment of the present invention provides the bis-[4-oxo-4-[3-(trifluoromethyl)-5,6-dihydro[1,2,4]triazolo[4,3-a]pyrazin-7(8H)-yl]-1-(2,4,5-trifluorophenyl)-butan-2-amine]phosphoric acid trihydrate salt of structural formula III:

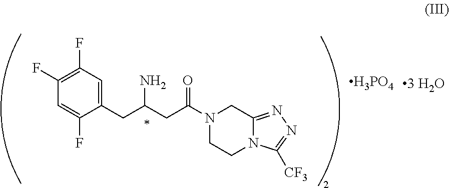

or a polymorph and/or solvate thereof.

The salt of structural formula I is comprised of two molar equivalents of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of phosphoric acid (H₃PO₄), and three molar equivalents of water.

Another embodiment of the present invention provides the bis(sitagliptin) monohydrogen phosphate trihydrate salt, which corresponds to the bis-[(2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid trihydrate salt of structural formula II-a:

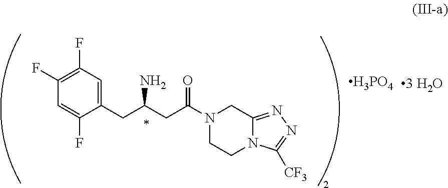

or a polymorph and/or solvate thereof.

The bis(sitagliptin) phosphoric acid trihydrate salt of structural formula III-a is comprised of two molar equivalents of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of phosphoric acid (H₃PO₄), and three molar equivalents of water.

Another embodiment of the present invention provides the bis-[(2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid trihydrate salt of structural formula III-b:

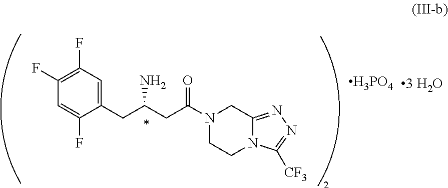

or a polymorph and/or solvate thereof.

The salt of structural formula III-b is comprised of two molar equivalents of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of phosphoric acid (H₃PO₄), and three molar equivalents of water.

Another class of this embodiment of the present invention provides the phosphoric acid salt drug substance of structural formulae III, III-a and III-b. Another class of this embodiment of the present invention provides the phosphoric acid salt drug substance of structural formula III, III-a and III-b that comprises the crystalline trihydrate present in a detectable amount. By “drug substance” is meant the active pharmaceutical ingredient (“API”). The amount of crystalline trihydrate in the drug substance can be quantified by the use of physical methods such as X-ray powder diffraction, solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy or any other technique. In a class of this embodiment, about 1% to about 100% by weight of the crystalline trihydrate is present in the drug substance. In a second class of this embodiment, about 5% to about 100% by weight of the crystalline trihydrate is present in the drug substance. In a third class of this embodiment, about 10% to about 100% by weight of the crystalline trihydrate is present in the drug substance. In a fourth class of this embodiment, about 25% to about 100% by weight of the crystalline trihydrate is present in the drug substance. In a fifth class of this embodiment, about 50% to about 100% by weight of the crystalline trihydrate is present in the drug substance. In a sixth class of this embodiment, about 75% to about 100% by weight of the crystalline trihydrate is present in the drug substance. In a seventh class of this embodiment, substantially all of the salt of structural formulae III, III-a and III-b drug substance is the crystalline trihydrate of the present invention, i.e., the salt of structural formulae III, III-a and III-b drug substance is substantially phase pure trihydrate.

Another embodiment of the present invention provides the bis-[4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid monohydrate salt of structural formula IV:

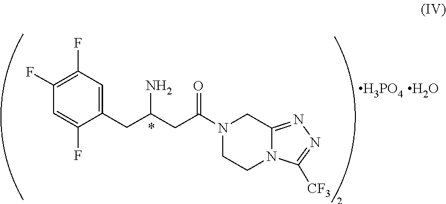

or a polymorph and/or solvate thereof.

The salt of structural formula IV is comprised of two molar equivalents of 4-oxo-4-[3-(trifluoromethyl)-5,6-dihydro[1,2,4]-triazolo[4,3-a]pyrazin-7(8H)-yl]-1-(2,4,5-trifluorophenyl)butan-2-amine, one molar equivalent of phosphoric acid (H₃PO₄), and one molar equivalent of water.

Another embodiment of the present invention provides the bis(sitagliptin) phosphoric acid monohydrate salt, which corresponds to the bis-[(2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid monohydrate salt of structural formula IV-a:

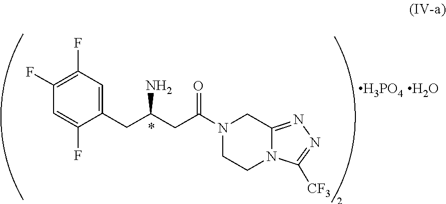

or a polymorph and/or solvate thereof.

The bis(sitagliptin) phosphoric acid monohydrate salt of structural formula IV-a is comprised of two molar equivalents of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of phosphoric acid (H₃PO₄), and one molar equivalent of water.

Another embodiment of the present invention provides the bis-[(2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid monohydrate salt of structural formula IV-b:

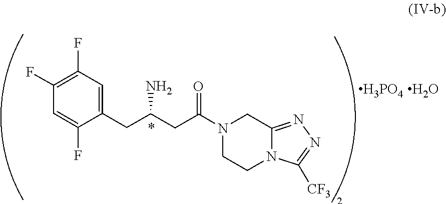

or a polymorph and/or solvate thereof.

The salt of structural formula IV-b is comprised of two molar equivalents of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of phosphoric acid (H₃PO₄), and one molar equivalent of water.

Another class of this embodiment of the present invention provides the phosphoric acid salt drug substance of structural formulae IV, IV-a and IV-b. Another class of this embodiment of the present invention provides the phosphoric acid salt drug substance of structural formulae IV, IV-a and IV-b that comprises the crystalline monohydrate present in a detectable amount. By “drug substance” is meant the active pharmaceutical ingredient (“API”). The amount of crystalline monohydrate in the drug substance can be quantified by the use of physical methods such as X-ray powder diffraction, solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy or any other technique. In a class of this embodiment, about 1% to about 100% by weight of the crystalline monohydrate is present in the drug substance. In a second class of this embodiment, about 5% to about 100% by weight of the crystalline monohydrate is present in the drug substance. In a third class of this embodiment, about 10% to about 100% by weight of the crystalline monohydrate is present in the drug substance. In a fourth class of this embodiment, about 25% to about 100% by weight of the crystalline monohydrate is present in the drug substance. In a fifth class of this embodiment, about 50% to about 100% by weight of the crystalline monohydrate is present in the drug substance. In a sixth class of this embodiment, about 75% to about 100% by weight of the crystalline monohydrate is present in the drug substance. In a seventh class of this embodiment, substantially all of the salt of structural formulae IV, IV-a and IV-b drug substance is the crystalline monohydrate of the present invention, i.e., the formulae IV, IV-a and IV-b salt drug substance is substantially phase pure monohydrate.

Another embodiment of the present invention provides the ammonia phosphoric acid salt of 4-oxo-4-[3-(trifluoromethyl)-5,6-dihydro[1,2,4]triazolo[4,3-a]pyrazin-7(8)-yl]-1-(2,4,5-trifluorophenyl)butan-2-amine of structural formula V:

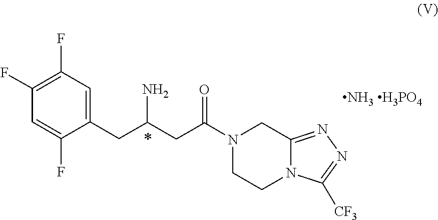 [SMILES: NC(CC(=O)N1CCn2c(nnc2C(F)(F)F)C1)Cc1cc(F)c(F)cc1F]

or a polymorph, hydrate and/or solvate thereof.

The salt of structural formula V is comprised of one molar equivalent of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of ammonia (NH₃), and one molar equivalent of phosphoric acid (H₃PO₄).

Another embodiment of the present invention provides the sitagliptin ammonia phosphoric acid salt, which corresponds to the ammonia phosphoric acid salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula V-a:

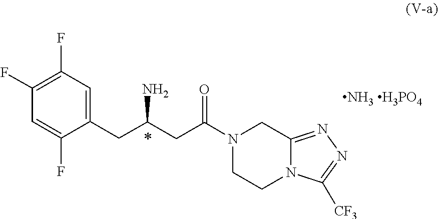 [SMILES: N[C@@H](CC(=O)N1CCn2c(nnc2C(F)(F)F)C1)Cc1cc(F)c(F)cc1F]

or a polymorph, hydrate and/or solvate thereof.

The sitagliptin ammonia phosphoric acid salt of structural formula V-a is comprised of one molar equivalent of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of ammonia (NH₃), and one molar equivalent of phosphoric acid (H₃PO₄).

Another embodiment of the present invention provides the ammonia phosphoric acid salt of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula V-b:

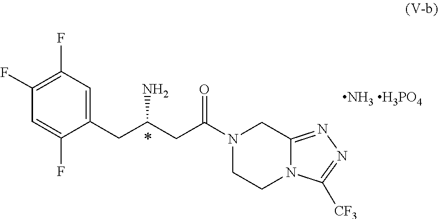 [SMILES: N[C@H](CC(=O)N1CCn2c(nnc2C(F)(F)F)C1)Cc1cc(F)c(F)cc1F]

or a polymorph, hydrate and/or solvate thereof.

The salt of structural formula V-b is comprised of one molar equivalent of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of ammonia (NH₃), and one molar equivalent of phosphoric acid (H₃PO₄).

Another embodiment of the present invention provides the ammonia phosphoric acid 2.5 hydrate salt of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula VI:

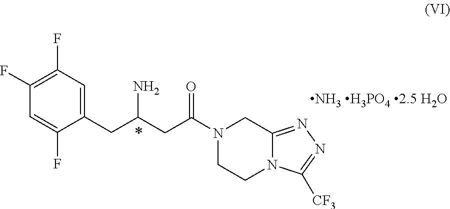 [SMILES: NC(CC(=O)N1CCn2c(nnc2C(F)(F)F)C1)Cc1cc(F)c(F)cc1F]

or a polymorph and/or solvate thereof.

The salt of structural formula VI is comprised of one molar equivalent of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of ammonia (NH₃), one molar equivalent of phosphoric acid (H₃PO₄), and 2.5 molar equivalents of water.

Another embodiment of the present invention provides the sitagliptin ammonia phosphoric acid 2.5 hydrate salt, which corresponds to the ammonia phosphoric acid 2.5 hydrate salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula VI-a:

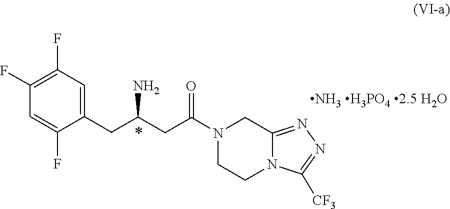 [SMILES: N[C@@H](CC(=O)N1CCn2c(nnc2C(F)(F)F)C1)Cc1cc(F)c(F)cc1F]

or a polymorph and/or solvate thereof.

The sitagliptin ammonia phosphoric acid 2.5 hydrate salt of structural formula VI-a is comprised of one molar equivalent of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of ammonia (NH₃), one molar equivalent of phosphoric acid (H₃PO₄), and 2.5 molar equivalents of water.

Another embodiment of the present invention provides the ammonia phosphoric acid 2.5 hydrate salt of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula VI-b:

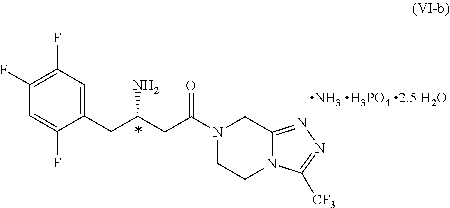 [SMILES: N[C@H](CC(=O)N1CCn2c(nnc2C(F)(F)F)C1)Cc1cc(F)c(F)cc1F]

or a polymorph and/or solvate thereof.

The salt of structural formula VI-b is comprised of one molar equivalent of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, one molar equivalent of ammonia (NH₃), one molar equivalent of phosphoric acid (H₃PO₄), and 2.5 molar equivalents of water.

Another class of this embodiment of the present invention provides the phosphoric acid salt drug substance of structural formulae VI, VI-a and VI-b. Another class of this embodiment of the present invention provides the phosphoric acid salt drug substance of structural formulae VI, VI-a and VI-b that comprises the crystalline 2.5 hydrate present in a detectable amount. By “drug substance” is meant the active pharmaceutical ingredient (“API”). The amount of crystalline 2.5 hydrate in the drug substance can be quantified by the use of physical methods such as X-ray powder diffraction, solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy or any other technique. In a class of this embodiment, about 1% to about 100% by weight of the crystalline 2.5 hydrate is present in the drug substance. In a second class of this embodiment, about 5% to about 100% by weight of the crystalline 2.5 hydrate is present in the drug substance. In a third class of this embodiment, about 10% to about 100% by weight of the crystalline 2.5 hydrate is present in the drug substance. In a fourth class of this embodiment, about 25% to about 100% by weight of the crystalline 2.5 hydrate is present in the drug substance. In a fifth class of this embodiment, about 50% to about 100% by weight of the crystalline 2.5 hydrate is present in the drug substance. In a sixth class of this embodiment, about 75% to about 100% by weight of the crystalline 2.5 hydrate is present in the drug substance. In a seventh class of this embodiment, substantially all of the salt of structural formulae VI, VI-a and VI-b drug substance is the crystalline 2.5 hydrate of the present invention, i.e., the formulae VI, VI-a and VI-b salt drug substance is substantially phase pure crystalline 2.5 hydrate.

Another embodiment of the present invention provides the bis(phosphoric acid) salt of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula VII:

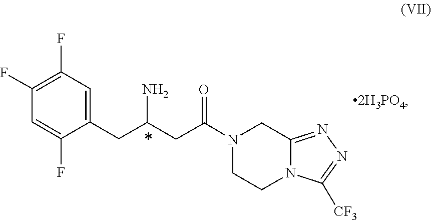 [SMILES: NC(CC(=O)N1CCn2c(nnc2C(F)(F)F)C1)Cc1cc(F)c(F)cc1F]

or a polymorph, hydrate and/or solvate thereof.

The salt of structural formula VII is comprised of one molar equivalent of 4-oxo-4-[3-(trifluoromethyl)-5,6-dihydro[1,2,4]triazolo[4,3-a]pyrazin-7(81H)-yl]-1-(2,4,5-trifluorophenyl)butan-2-amine, and two molar equivalents of phosphoric acid (H₃PO₄).

Another embodiment of the present invention provides the sitagliptin bis(phosphoric acid) salt, which corresponds to the bis(phosphoric acid) salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula VII-a:

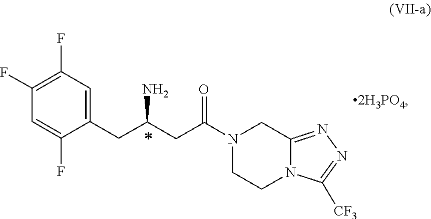 [SMILES: N[C@@H](CC(=O)N1CCn2c(nnc2C(F)(F)F)C1)Cc1cc(F)c(F)cc1F]

or a polymorph, hydrate and/or solvate thereof.

The sitagliptin bis(phosphoric acid) salt of structural formula VII-a is comprised of one molar equivalent of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, and two molar equivalents of phosphoric acid (H₃PO₄).

Another embodiment of the present invention provides the bis(phosphoric acid) salt of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula VII-b:

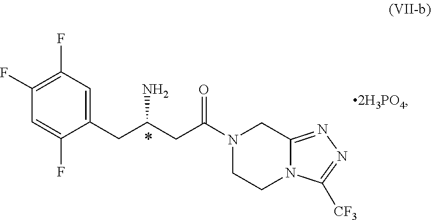 [SMILES: N[C@H](CC(=O)N1CCn2c(nnc2C(F)(F)F)C1)Cc1cc(F)c(F)cc1F]

or a polymorph, hydrate and/or solvate thereof.

The salt of structural formula VII-b is comprised of one molar equivalent of (2S)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, and two molar equivalents of phosphoric acid (H₃PO₄).

Another class of this embodiment of the present invention provides the bis(phosphoric acid) salt drug substance of structural formulae VII, VII-a and VII-b. Another class of this embodiment of the present invention provides the bis(phosphoric acid) salt drug substance of structural formulae VII, VII-a and VII-b that comprises the amorphous bis(phosphoric acid) salt present in a detectable amount. Another class of this embodiment of the present invention provides the bis(phosphoric acid) salt drug substance of structural formulae VII, VII-a and VII-b that comprises the crystalline bis(phosphoric acid) salt present in a detectable amount. By “drug substance” is meant the active pharmaceutical ingredient (“API”). The amount of crystalline bis(phosphoric acid) salt in the drug substance can be quantified by the use of physical methods such as X-ray powder diffraction, solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy or any other technique. In a class of this embodiment, about 1% to about 100% by weight of the crystalline bis(phosphoric acid) salt is present in the drug substance. In a second class of this embodiment, about 5% to about 100% by weight of the crystalline bis(phosphoric acid) salt is present in the drug substance. In a third class of this embodiment, about 10% to about 100% by weight of the crystalline bis(phosphoric acid) salt is present in the drug substance. In a fourth class of this embodiment, about 25% to about 100% by weight of the crystalline bis(phosphoric acid) salt is present in the drug substance. In a fifth class of this embodiment, about 50% to about 100% by weight of the crystalline bis(phosphoric acid) salt is present in the drug substance. In a sixth class of this embodiment, about 75% to about 100% by weight of the crystalline bis(phosphoric acid) salt is present in the drug substance. In a seventh class of this embodiment, substantially all of the salt of structural formulae VII, VII-a and VII-b drug substance is the crystalline bis(phosphoric acid) salt of the present invention, i.e., the formulae VII, VII-a and VII-b salt drug substance is substantially phase pure crystalline bis(phosphoric acid) salt. In a eighth class of this embodiment, about 1% to about 100% by weight of the amorphous bis(phosphoric acid) salt is present in the drug substance. In a ninth class of this embodiment, about 5% to about 100% by weight of the amorphous bis(phosphoric acid) salt is present in the drug substance. In a tenth class of this embodiment, about 10% to about 100% by weight of the amorphous bis(phosphoric acid) salt is present in the drug substance. In a eleventh class of this embodiment, about 25% to about 100% by weight of the amorphous bis(phosphoric acid) salt is present in the drug substance. In a twelfth class of this embodiment, about 50% to about 100% by weight of the amorphous bis(phosphoric acid) salt is present in the drug substance. In a thirteenth class of this embodiment, about 75% to about 100% by weight of the amorphous bis(phosphoric acid) salt is present in the drug substance. In a fourteenth class of this embodiment, substantially all of the salt of structural formulae VII, VII-a and VII-b drug substance is the amorphous bis(phosphoric acid) salt of the present invention, i.e., the formulae VII, VII-a and VII-b salt drug substance is substantially phase pure amorphous bis(phosphoric acid) salt.

In a further embodiment of the present invention, the phosphoric acid salts of structural formulae I-VII are amorphous.

In a further embodiment of the present invention, the phosphoric acid salts of structural formulae I-VII are crystalline or polymorphs.

In a further embodiment of the present invention, the phosphoric acid salts of structural formulae I-VII are anhydrous.

In a further embodiment of the present invention, the phosphoric acid salts of structural formulae I-VII are hydrates.

In a further embodiment of the present invention, the phosphoric acid salts of structural formulae I-VII are crystalline hydrates.

The term “formulae I-VII” includes formulae I, I-a, I-b, II, II-a, II-b, I, III-a, III-b, IV, IV-a, IV-b, V, V-a, V-b, VI, VI-a, VI-b, VII, VII-a, and VII-b.

The term “sitagliptin” in the present invention means “(2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine”.

The terms “bi” and “bis” in the present invention mean two.

The terms “bi-sitagliptin” and “bis(sitagliptin)” in the present invention mean two molecules of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine.

The terms “bi-phosphoric acid” and “bis(phosphoric acid)” in the present invention mean two molecules of phosphoric acid (H₃PO₄).

The term “hydrate” is meant to include all full, multiple and partial hydrates of the salts of the compounds of formulae I-VII, including, but not limited to, the mono-hydrate, hemi-hydrate, bis-hydrate, 2.5 hydrate, and trihydrate.

The term “solvate” is meant to include compound forms containing solvent molecules within the crystal structure of salts of the present invention, including but not limited to, methanol, ethanol, isopropanol and acetone.

The term “% enantiomeric excess” (abbreviated “ee”) shall mean the % major enantiomer less the % minor enantiomer. Thus, a 70% enantiomeric excess corresponds to formation of 85% of one enantiomer and 15% of the other. The term “enantiomeric excess” is synonymous with the term “optical purity.”

The phosphoric acid salts of the present invention exhibit pharmaceutic advantages over the free base and the previously disclosed hydrochloride salt (WO 03/004498) in the preparation of a pharmaceutical drug product containing the pharmacologically active ingredient. In particular, the enhanced physical stability of the phosphoric acid salts of the present invention constitutes one example of a surprising and useful property that can be taken advantage of in the preparation of solid pharmaceutical dosage forms containing the pharmacologically active ingredient. Specifically, the bis(sitagliptin) phosphoric acid trihydrate salt is unexpectedly stable at elevated temperature and humidity.

The phosphoric acid salts of the present invention, which exhibit potent DP-IV inhibitory properties, are particularly useful for the prevention or treatment of Type 2 diabetes, obesity, and high blood pressure.

Another aspect of the present invention provides a method for the prevention or treatment of clinical conditions for which an inhibitor of DP-IV is indicated, which method comprises administering to a patient in need of such prevention or treatment a prophylactically or therapeutically effective amount of a phosphoric acid salt of the compound of structural formula I or a polymorph, hydrate and/or solvate thereof. Such clinical conditions include diabetes, in particular Type 2 diabetes, hyperglycemia, insulin resistance, and obesity.

The present invention also provides the use of the phosphoric acid salts of the compound of structural formula I or a polymorph, hydrate and/or solvate thereof, for the manufacture of a medicament for the prevention or treatment of clinical conditions for which an inhibitor of DP-IV is indicated.

The present invention also provides pharmaceutical compositions comprising a phosphoric acid salt of the compound of structural formula I or a polymorph, hydrate and/or solvate thereof, in association with one or more pharmaceutically acceptable carriers or excipients.

The compositions in accordance with the invention are suitably in unit dosage forms such as tablets, pills, capsules, powders, granules, sterile solutions or suspensions, metered aerosol or liquid sprays, drops, ampoules, auto-injector devices or suppositories. The compositions are intended for oral, parenteral, intranasal, sublingual, or rectal administration, i.v. administration, or for administration by inhalation or insufflation.

The dosage regimen is selected in accordance with a variety of factors including type, species, age, weight, sex and medical condition of the patient; the severity of the condition to be treated; the route of administration; and the renal and hepatic function of the patient. An ordinarily skilled physician, veterinarian, or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, 200, and 500 milligrams of the active ingredient for the symptomatic adjustment of the dosage to the patient to be treated. A medicament typically contains from about 0.01 mg to about 500 mg of the active ingredient, preferably, from about 1 mg to about 200 mg of active ingredient. Intravenously, the most preferred doses will range from about 0.1 to about 10 mg/kg/minute during a constant rate infusion. Advantageously, the salts of the present invention may be administered in a single daily dose, or the total daily dosage may be administered in divided doses of two, three or four times daily. Furthermore, the salts of the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the present invention, the phosphoric acid salts of the compound of formula I, and polymorphs, hydrates and/or solvates thereof, herein described in detail can form the active pharmaceutical ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active pharmaceutical ingredient can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the active pharmaceutical ingredient can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

According to a further aspect, the present invention provides a process for the preparation of the trihydrate salt of formula III, which process comprises reacting approximately two equivalents of the free base of 4-oxo-4-[3-(trifluoromethyl)-5,6-dihydro-[1,2,4]triazolo[4,3-a]pyrazin-7(8H)-yl]-1-(2,4,5-trifluorophenyl)butan-2-amine of structural formula I below:

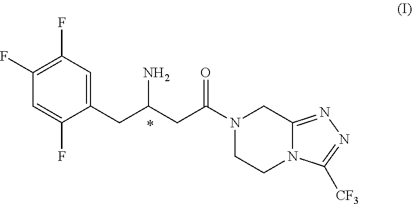 [SMILES: NC(CC(=O)N1CCn2c(nnc2C(F)(F)F)C1)Cc1cc(F)c(F)cc1F]

with approximately one equivalent of phosphoric acid in a suitable aqueous C₁-C₅ alkanol, such as methanol, ethanol, or isopropyl alcohol (IPA). The reaction is carried out at a temperature range of about 25° C. to about 80° C. The phosphoric acid solution is added to the solution of the amine. The solution is heated, and then stirred at room temperature. The resulting solid is dried to give the crystalline bis-[4-oxo-4-[3-(trifluoromethyl)-5,6-dihydro-[1,2,4]triazolo[4,3-a]pyrazin-7(8H)-yl]-1-(2,4,5-trifluorophenyl)butan-2-amine]phosphoric acid trihydrate salt.

According to a further aspect, the present invention provides a process for the preparation of the monohydrate salt of structural formula IV, which process comprises reacting approximately two equivalents of the free base of 4-oxo-4-[3-(trifluoromethyl)-5,6-dihydro-[1,2,4]triazolo[4,3-a]pyrazin-7(8H)-yl]-1-(2,4,5-trifluorophenyl)butan-2-amine of structural formula I below:

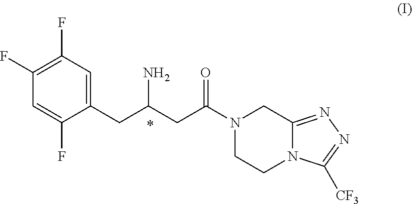 [SMILES: NC(CC(=O)N1CCn2c(nnc2C(F)(F)F)C1)Cc1cc(F)c(F)cc1F]

with approximately one equivalent of phosphoric acid in a suitable C₁-C₅ alkanol, such as methanol, ethanol, or isopropyl alcohol (IPA). The reaction is carried out at a temperature range of about 25° C. to about 80° C. The phosphoric acid solution is added to the solution of the amine. The reaction is heated, then stirred at room temperature. The resulting solid is dried to give the crystalline bi-[4-oxo-4-[3-(trifluoromethyl)-5,6-dihydro-[1,2,4]triazolo[4,3-a]pyrazin-7(8H)-yl]-1-(2,4,5-trifluorophenyl)butan-2-amine]phosphoric acid monohydrate salt.

According to a further aspect, the present invention provides a process for the preparation of the 2.5 hydrate salt of formula V, which process comprises reacting approximately one equivalent of the free base of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula I below:

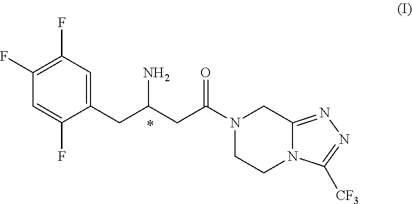 [SMILES: NC(CC(=O)N1CCn2c(nnc2C(F)(F)F)C1)Cc1cc(F)c(F)cc1F]

with approximately one equivalent of ammonium phosphate in a suitable aqueous C₁-C₅ alkanol, such as methanol, ethanol, or isopropyl alcohol (IPA). The reaction is carried out at a temperature range of about 25° C. to about 80° C. The ammonium phosphate solution is added to the solution of the amine. The reaction was heated, and then cooled to room temperature. The resulting solid is dried to give the 4-oxo-4-[3-(trifluoromethyl)-5,6-dihydro-[1,2,4]triazolo[4,3-a]pyrazin-7(8H)-yl]-1-(2,4,5-trifluorophenyl)butan-2-amine ammonia phosphoric acid 2.5 hydrate salt.

According to a further aspect, the present invention provides a process for the preparation of the bis(phosphoric acid) salt of formula VII, which process comprises reacting approximately one equivalent of the free base of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine of structural formula I below:

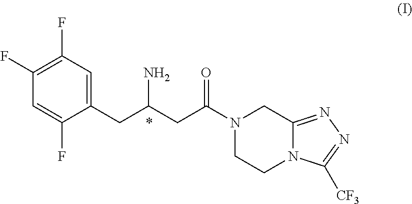 [SMILES: NC(CC(=O)N1CCn2c(nnc2C(F)(F)F)C1)Cc1cc(F)c(F)cc1F]

with approximately two equivalents of phosphoric acid in acetone. The reaction is carried out at a temperature range of about 25° C. to about 80° C. The phosphoric acid is added to the solution of the amine. The reaction was heated, and then cooled to room temperature. The resulting solid is dried to give the 4-oxo-4-[3-(trifluoromethyl)-5,6-dihydro-[1,2,4]triazolo[4,3-a]pyrazin-7(8H)-yl]-1-(2,4,5-trifluorophenyl)butan-2-amine bis(phosphoric acid) salt.

The starting compound of structural formula I can be prepared by the procedures detailed in Schemes 1-3 and Reference Example 1 below.

In a still further aspect, the present invention provides a method for the treatment and/or prevention of clinical conditions for which a DP-IV inhibitor is indicated, which method comprises administering to a patient in need of such prevention or treatment a prophylactically or therapeutically effective amount of the salt of Formula I as defined above or a polymorph, hydrate and/or solvate thereof.

The following non-limiting Examples are intended to illustrate the present invention and should not be construed as being limitations on the scope or spirit of the instant invention.

Compounds described herein may exist as tautomers such as keto-enol tautomers. The individual tautomers as well as mixtures thereof are encompassed with compounds of structural formulae I-VII.

Compounds described herein may exist as polymorphs. Polymorphs are compounds having the same chemical composition but different crystalline (or crystal) structures. Polymorphism is the ability of the same chemical substance to exist as different crystalline structures.

In the schemes, reference example and examples below, various reagent symbols and abbreviations have the following meanings: ACN is acetonitrile; CD₃CN is deuterated acetonitrile; g is gram(s); kg is kilogram(s); IPA is isopropanol; IPAC is isopropyl acetate; L is liter(s); mol is mole(s); mL is milliliter; MeOH is methanol; m.p. is melting point; MTBE is methyl tert-butyl ether; N is normal; w/w is by weight; wt % is weight percent; iPr₂NEt is diisopropyl ethyl amine; DMAP is 4-(dimethylamino)pyridine; DMAc is N,N-dimethylacetamide; [Rh(cod)Cl]₂ is chloro(1,5-cyclooctadiene)rhodium(I) dimer; t-Bu is tert-butyl; and h is hour(s).

### Reference Example 1

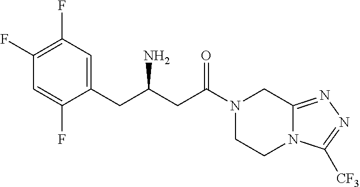 [SMILES: N[C@@H](CC(=O)N1CCn2c(nnc2C(F)(F)F)C1)Cc1cc(F)c(F)cc1F]

### (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine (sitagliptin free base)

### Preparation of 3-(trifluoromethyl)-5,6,7,8-tetrahydro[1,2,4]triazolo[4,3-a]pyrazine hydrochloride (1-4)

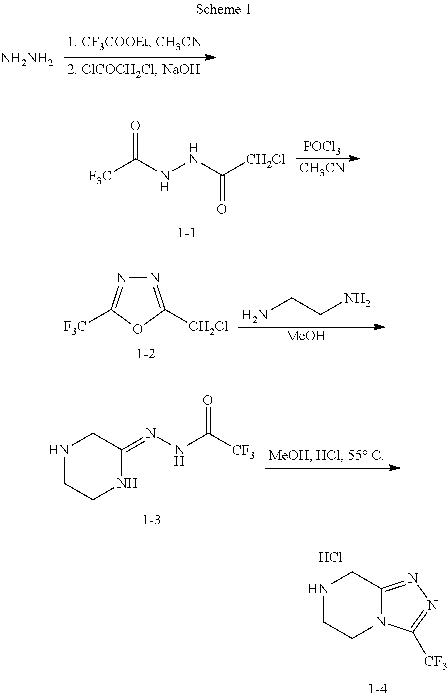

### Step A:

### Preparation of Bishydrazide (1-1)

Hydrazine (20.1 g, 35 wt % in water, 0.22 mol) was mixed with 310 mL of acetonitrile. 31.5 g of ethyl trifluoroacetate (0.22 mol) was added over 60 min. The internal temperature was increased to 25° C. from 14° C. The resulting solution was aged at 22-25° C. for 60 min. The solution was cooled to 7° C. 17.9 g of 50 wt % aqueous NaOH (0.22 mol) and 25.3 g of chloroacetyl chloride (0.22 mol) were added simultaneously over 130 min at a temperature below 16° C. When the reaction was complete, the mixture was vacuum distilled to remove water and ethanol at 27˜30° C. and under 26˜27 in Hg vacuum. During the distillation, 720 mL of acetonitrile was added slowly to maintain constant volume (approximately 500 mL). The slurry was filtered to remove sodium chloride. The cake was rinsed with about 100 mL of acetonitrile. Removal of the solvent afforded bis-hydrazide 1-1 (43.2 g, 96.5% yield, 94.4 area % pure by HPLC assay). ¹H-NMR (400 MHz, DMSO-d₆): δ 4.2 (s, 2H), 10.7 (s, 1H), and 11.6 (s, 1H) ppm. ¹³C-NMR (100 MHz, DMSO-d₆): δ 41.0, 116.1 (q, J=362 Hz), 155.8 (q, J=50 Hz), and 165.4 ppm.

### Step B:

### Preparation of 5-(trifluoromethyl)-2-(chloromethyl)-1,3,4-oxadiazole (1-2)

Bishydrazide 1-1 from Step A (43.2 g, 0.21 mol) in ACN (82 mL) was cooled to 5° C. Phosphorus oxychloride (32.2 g, 0.21 mol) was added, maintaining the temperature below 10° C. The mixture was heated to 80° C. and aged at this temperature for 24 h until HPLC showed less than 2 area % of 1-1. In a separate vessel, 260 mL of IPAc and 250 mL of water were mixed and cooled to 0° C. The reaction slurry was charged to the quench keeping the internal temperature below 10° C. After the addition, the mixture was agitated vigorously for 30 min, the temperature was increased to room temperature and the aqueous layer was cut. The organic layer was then washed with 215 mL of water, 215 mL of 5 wt % aqueous sodium bicarbonate and finally 215 mL of 20 wt % aqueous brine solution. HPLC assay yield after work up was 86-92%. Volatiles were removed by distillation at 75-80 mm Hg, 55° C. to afford an oil which could be used directly in Step C without further purification. Otherwise the product can be purified by distillation to afford 1-2 in 70-80% yield. ¹H-NMR (400 MHz, CDCl₃): δ 4.8 (s, 2H) ppm.

¹³C-NMR (100 MHz, CDCl₃): δ 32.1, 115.8 (q, J=337 Hz), 156.2 (q, J=50 Hz), and 164.4 ppm.

### Step C:

### Preparation of N-[(2Z)-piperazin-2-ylidene]trifluoroacetohydrazide (1-3)

To a solution of ethylenediamine (33.1 g, 0.55 mol) in methanol (150 mL) cooled at −20° C. was added distilled oxadiazole 1-2 from Step B (29.8 g, 0.16 mol) while keeping the internal temperature at −20° C. After the addition was complete, the resulting slurry was aged at −20° C. for 1 h. Ethanol (225 mL) was then charged and the slurry slowly warmed to −5° C. After 60 min at −5° C., the slurry was filtered and washed with ethanol (60 mL) at −5° C. Amidine 1-3 was obtained as a white solid in 72% yield (24.4 g, 99.5 area wt % pure by HPLC). ¹H-NMR (400 MHz, DMSO-d₆): δ 2.9 (t, 2H), 3.2 (t, 2H), 3.6 (s, 2H), and 8.3 (b, 1H) ppm. ¹³C-NMR (100 MHz, DMSO-d₆): δ 40.8, 42.0, 43.3, 119.3 (q, J=350 Hz), 154.2, and 156.2 (q, J=38Hz) ppm.

### Step D:

### Preparation of 3-(trifluoromethyl)-5,6,7,8-tetrahydro[1,2,4]triazolo[4,3-a]pyrazine hydrochloride (1-4)

A suspension of amidine 1-3 (27.3 g, 0.13 mol) in 110 mL of methanol was warmed to 55° C. 37% Hydrochloric acid (11.2 mL, 0.14 mol) was added over 15 min at this temperature. During the addition, all solids dissolved resulting in a clear solution. The reaction was aged for 30 min. The solution was cooled down to 20° C. and aged at this temperature until a seed bed formed (10 min to 1 h). 300 mL of MTBE was charged at 20° C. over 1 h. The resulting slurry was cooled to 2° C., aged for 30 min and filtered. Solids were washed with 50 mL of ethanol:MTBE (1:3) and dried under vacuum at 45° C. Yield of triazole 1-4 was 26.7 g (99.5 area wt % pure by HPLC).

¹H-NMR (400 MHz, DMSO-d₆): δ 3.6 (t, 2H), 4.4 (t, 2H), 4.6 (s, 2H), and 10.6 (b, 2H) ppm;

¹³C-NMR (100 MHz, DMSO-d₆): δ: 39.4, 39.6, 41.0, 118.6 (q, J=325 Hz), 142.9 (q, J=50 Hz), and 148.8 ppm.

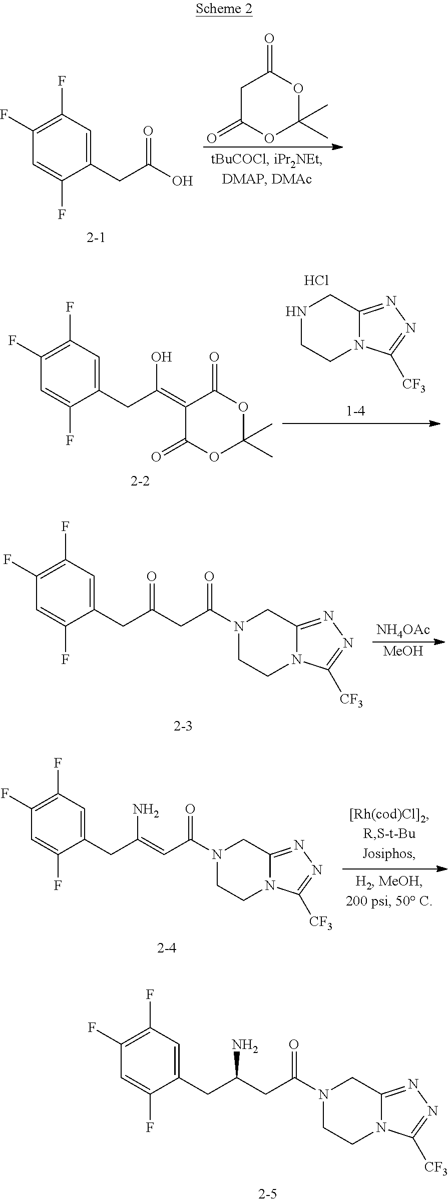

### Step A:

### Preparation of 4-oxo-4-[3-(trifluoromethyl)-5,6-dihydro[1,2,4]triazolo[4,3-a]pyrazin-7(8H)-yl]-1-(2,4,5-trifluorophenl)butan-2-one (2-3)

2,4,5-Trifluorophenylacetic acid (2-1) (150 g, 0.789 mol), Meldrum's acid (125 g, 0.868 mol), and 4-(dimethylamino)pyridine (DMAP) (7.7 g, 0063 mol) were charged into a 5 L three-neck flask. N,N-Dimethylacetamide (DMAc) (525 mL) was added in one portion at room temperature to dissolve the solids. N,N-diisopropylethylamine (282 mL, 1.62 mol) was added in one portion at room temperature while maintaining the temperature below 40° C. Pivaloyl chloride (107 mL, 0.868 mol) was added dropwise over 1 to 2 h while maintaining the temperature between 0 and 5° C. The reaction mixture was aged at 5° C. for 1 h. Triazole hydrochloride 1-4 (180 g, 0.789 mol) was added in one portion at 40-50° C. The reaction solution was aged at 70° C. for several h. 5% Aqueous sodium hydrogencarbonate solution (625 mL) was then added dropwise at 20-45° C. The batch was seeded and aged at 20-30° C. for 1-2 h. Then an additional 525 mL of 5% aqueous sodium hydrogencarbonate solution was added dropwise over 2-3 h. After aging several h at room temperature, the slurry was cooled to 0-5° C. and aged 1 h before filtering the solid. The wet cake was displacement-washed with 20% aqueous DMAc (300 mL), followed by an additional two batches of 20% aqueous DMAc (400 mL), and finally water (400 mL). The cake was suction-dried at room temperature. The isolated yield of final product 2-3 was 89%.

### Step B:

### Preparation of (2Z)-4-oxo-4-[3-(trifluoromethyl)-5,6-dihydro[1,2,4]triazolo[4,3-a]pyrazin-7(8H)-yl]-1-(2,4,5-trifluorophenyl)but-2-en-2-amine (2-4)

A 5 L round-bottom flask was charged with methanol (100 mL), the ketoamide 2-3 (200 g), and ammonium acetate (110.4 g). Methanol (180 mL) and 28% aqueous ammonium hydroxide (58.6 mL) were then added keeping the temperature below 30° C. during the addition. Additional methanol (100 mL) was added to the reaction mixture. The mixture was heated at reflux temperature and aged for 2 h. The reaction was cooled to room temperature and then to about 5° C. in an ice-bath. After 30 min, the solid was filtered and dried to afford 2-4 as a solid (180 g); m.p. 271.2° C.

### Step C:

### Preparation of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine (2-5)

Into a 500 ml flask were charged chloro(1,5-cyclooctadiene)rhodium(I) dimer {[Rh(cod)Cl]₂}(292 mg, 1.18 mmol) and (R,S) t-butyl Josiphos (708 mg, 1.3 mmol) under a nitrogen atmosphere. Degassed MeOH was then added (200 mL) and the mixture was stirred at room temperature for 1 h. Into a 4 L hydrogenator was charged the enamine amide 2-4 (118 g, 0.29 mol) along with MeOH (1 L). The slurry was degassed. The catalyst solution was then transferred to the hydrogenator under nitrogen. After degassing three times, the enamine amide was hydrogenated under 200 psi hydrogen gas at 50° C. for 13 h. Assay yield was determined by HPLC to be 93% and optical purity to be 94% ee.

The optical purity was further enhanced in the following manner. The methanol solution from the hydrogenation reaction (18 g in 180 mL MeOH) was concentrated and switched to methyl t-butyl ether (MTBE) (45 mL). Into this solution was added aqueous H₃PO₄ solution (0.5 M, 95 mL). After separation of the layers, 3N NaOH (35 mL) was added to the water layer, which was then extracted with MTBE (180 mL+100 mL). The MTBE solution was concentrated and solvent switched to hot toluene (180 mL, about 75° C.). The hot toluene solution was then allowed to cool to 0° C. slowly (5-10 h). The crystals were isolated by filtration (13 g, yield 72%, 98-99% ee); m.p. 114.1-115.7° C.

¹H NMR (300 MHz, CD₃CN): δ 7.26 (m), 7.08 (m), 4.90 (s), 4.89 (s), 4.14 (m), 3.95 (m), 3.40 (m), 2.68 (m), 2.49 (m), 1.40 (bs).

Compound 2-5 exists as amide bond rotamers. Unless indicated, the major and minor rotamers are grouped together since the carbon-13 signals are not well resolved:

¹³C NMR (CD₃CN): δ 171.8, 157.4 (ddd, J_{CF}=242.4, 9.2, 2.5 Hz), 152.2 (major), 151.8 (minor), 149.3 (ddd; J_{CF}=246.7, 14.2, 12.9 Hz), 147.4 (ddd, J_{CF}=241.2, 12.3, 3.7 Hz), 144.2 (q, J_{F}=38.8Hz), 124.6 (ddd, J_{CF}=18.5, 5.9, 4.0 Hz), 120.4 (dd, J_{F}=19.1, 6.2 Hz), 119.8 (q, J_{CF}=268.9 Hz), 106.2 (dd, J_{CF}=29.5, 20.9 Hz), 50.1, 44.8, 44.3 (minor), 43.2 (minor), 42.4, 41.6 (minor), 41.4, 39.6, 38.5 (minor), 36.9.

The crystalline sitagliptin free base can be isolated as follows:
(a) The reaction mixture upon completion of the hydrogenation step is charged with 25 wt % of Ecosorb™ C-941. The mixture is stirred under nitrogen for one hour and then filtered. The cake is washed with 2 L/kg of methanol. Recovery of free base is about 95% and optical purity about 95% ee.
(b) The freebase solution in methanol is concentrated to 3.5-4.0 L/kg volume (based on free base charge) and then solvent-switched into isopropanol (IPA) to final volume of 3.0 L/kg IPA.
(c) The slurry is heated to 40° C. and aged 1 h at 40° C. and then cooled to 25° C. over 2 h.
(d) Heptane (7 L/kg) is charged over 7 h and the slurry stirred for 12 h at 22-25° C. The supernatant concentration before filtering is 10-12 mg/g.
(e) The slurry is filtered and the solid washed with 30% IPA/heptane (2 L/kg).
(f) The solid is dried in a vacuum oven at 40° C.
(g) The optical purity of the free base is about 99% ee.

The following high-performance liquid chromatographic (HPLC) conditions were used to determine percent conversion to product:
Column: Waters Symmetry C18, 250 mm×4.6 mm
Eluent: Solvent A: 0.1 vol % HC104/H₂O

Solvent B: acetonitrile
Gradient: 0 min 75% A: 25% B

10 min 25% A: 75% B

12.5 min 25% A: 75% B

15 min 75% A: 25% B
Flow rate: 1 mL/min

### Injection Vol.: 10 μL

UV detection: 210 nm
Column temp.: 40° C.
Retention times: compound 2-4: 9.1 min
   compound 2-5: 5.4 min
   tBu Josiphos: 8.7 min

The following high-performance liquid chromatographic (HPLC) conditions were used to determine optical purity:
Column: Chirapak, AD-H, 250 mm×4.6 mm
Eluent: Solvent A: 0.2 vol. % diethylamine in heptane

Solvent B: 0.1 vol % diethylamine in ethanol

### Isochratic Run Time: 18 min

Flow rate: 0.7 mL/min

### Injection Vol.: 7 μL

UV detection: 268 nm
Column temp.: 35° C.
Retention times: (R)-amine 2-5: 13.8 min
   (S)-amine 2-5: 11.2 min

### Example 1

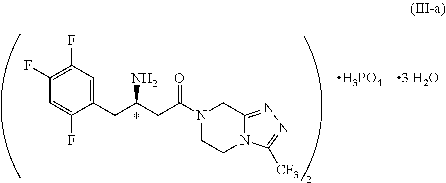

### Bis-[(2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid trihydrate salt (also known as the bis(sitagliptin) phosphoric acid trihydrate salt)

Sitagliptin free base (1.50 g, 0.00368 moles) was combined with isopropanol (3.2 mL) and distilled water (1.4 mL). The mixture was stirred for 5-10 minutes to form a clear solution. Phosphoric acid (85% w/w, 0.215 g, 0.00186 moles) was added with stirring. The solution was heated with stirring to 70° C. for 15 minutes, then cooled to room temperature and left stirring overnight. The solution solidified. The resulting solid was dried for approximately 6 hours at room temperature under vacuum to give the bis(sitagliptin) phosphoric acid trihydrate salt, which corresponds to [(sitagliptin)₂(H₃PO₄)(H₂O)₃] salt. Elemental analysis: C (39.36%), H (3.83%), N (14.30%), P (3.02%).

X-ray powder diffraction studies are widely used to characterize molecular structures, crystallinity, and polymorphism. The X-ray powder diffraction pattern of the crystalline trihydrate form of the bis(sitagliptin) phosphoric acid salt was generated on a Philips Analytical X'Pert PRO X-ray Diffraction System with PW3040/60 console. A PW3373/00 ceramic Cu LEF X-ray tube K-Alpha radiation was used as the source.

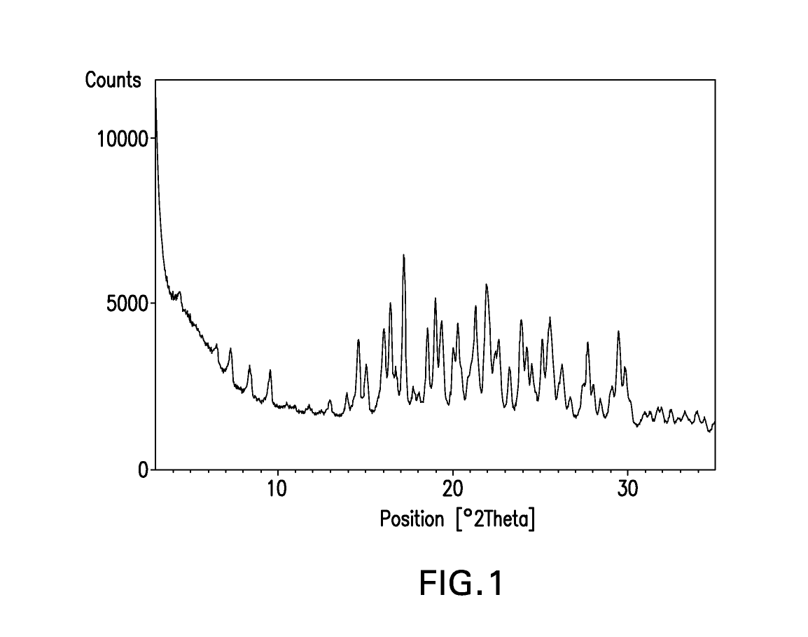

FIG. 1 shows the X-ray diffraction pattern for the crystalline trihydrate form of the bis(sitagliptin) phosphoric acid salt of structural formula III-a. The trihydrate exhibited diffraction peaks corresponding to d-spacings of 5.1, 4.0, and 20.1 angstroms. The trihydrate was further characterized by the d-spacings of 4.7, 4.2, and 5.4 angstroms. The trihydrate was even further characterized by the d-spacings of 3.5, 3.7, and 4.6 angstroms.

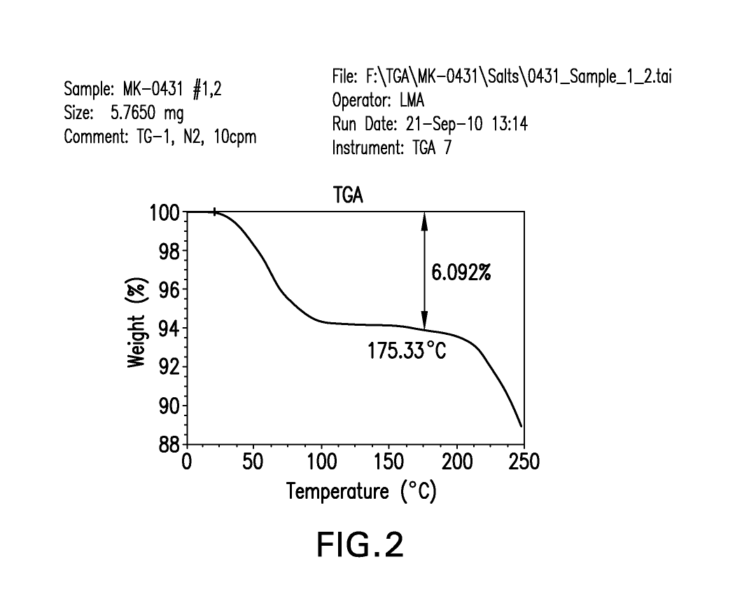

FIG. 2 shows the thermogravimetric analysis (TGA) curve for the crystalline trihydrate form of the bis(sitagliptin) phosphoric acid salt of structural formula III-a. TG data were acquired using a Perkin Elmer model TGA 7. Experiments were performed under a flow of nitrogen and using a heating rate of 10° C./min to a maximum temperature of approximately 250° C. After automatically taring the balance, 5 to 20 mg of sample was added to the platinum pan, the furnace was raised, and the heating program started. Weight/temperature data were collected automatically by the instrument. Analysis of the results was carried out by selecting the Delta Y function within the instrument software and choosing the temperatures between which the weight loss is to be calculated. Weight losses are reported up to the onset of decomposition/evaporation. TGA indicated a weight loss of about 6.092% from ambient temperature to about 175.33° C.

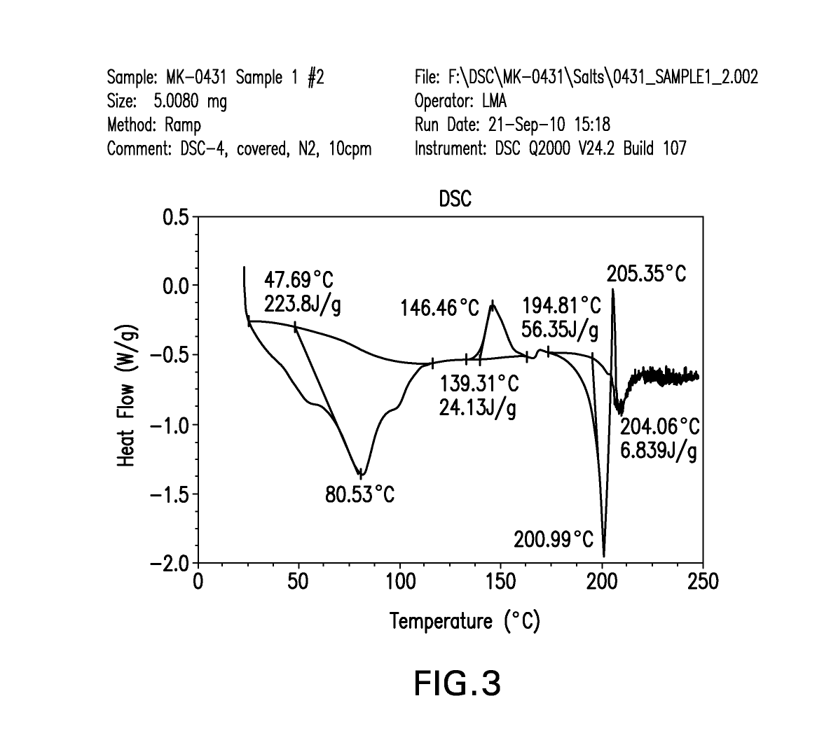

FIG. 3 shows the DSC curve for the crystalline trihydrate form of the bis(sitagliptin) phosphoric acid salt of structural formula III-a. DSC data were acquired using TA Instruments DSC Q2000 or equivalent. Between 2 and 6 mg sample was weighed into a pan and covered. This pan was then covered and placed at the sample position in the calorimeter cell. An empty pan was placed at the reference position. The calorimeter cell was closed and a flow of nitrogen was passed through the cell. The heating program was set to heat the sample at a heating rate of 10° C./min to a temperature of approximately 250° C. The heating program was started. When the run was completed, the data were analyzed using the DSC analysis program contained in the system software. The thermal events were integrated between baseline temperature points that are above and below the temperature range over which the thermal event was observed. The data reported were the onset temperature, peak temperature and enthalpy.

The crystalline bis(sitagliptin) phosphoric acid trihydrate salt was found to be physically stable at 40° C. at 75% relative humidity for 2 weeks.

### Example 2

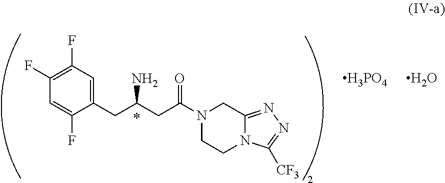

### Bis-[(2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid monohydrate salt (also known as bis(sitagliptin) phosphoric acid monohydrate salt)

Sitagliptin free base (1.50 g, 0.00368 moles) was combined with methanol (4.6 mL). Phosphoric acid (85% w/w, 0.21 g, 0.0018 moles) was added to the solution. The solution was heated with stirring to 70° C. for 15 minutes and then cooled to room temperature. A white crystalline powder formed. The resulting solid crystalline powder was dried overnight at room temperature under vacuum to give the bis(sitagliptin) phosphoric acid monohydrate salt, which corresponds to [(sitagliptin)₂(H₃PO₄)(H₂O)] salt. Elemental analysis: C (40.81%), H (3.54%), N (14.82%), P (3.39%).

X-ray powder diffraction studies are widely used to characterize molecular structures, crystallinity, and polymorphism. The X-ray powder diffraction pattern of the crystalline bis(sitagliptin) monohydrogen phosphate monohydrate salt was generated on a Philips Analytical X'Pert PRO X-ray Diffraction System with PW3040/60 console. A PW3373/00 ceramic Cu LEF X-ray tube K-Alpha radiation was used as the source.

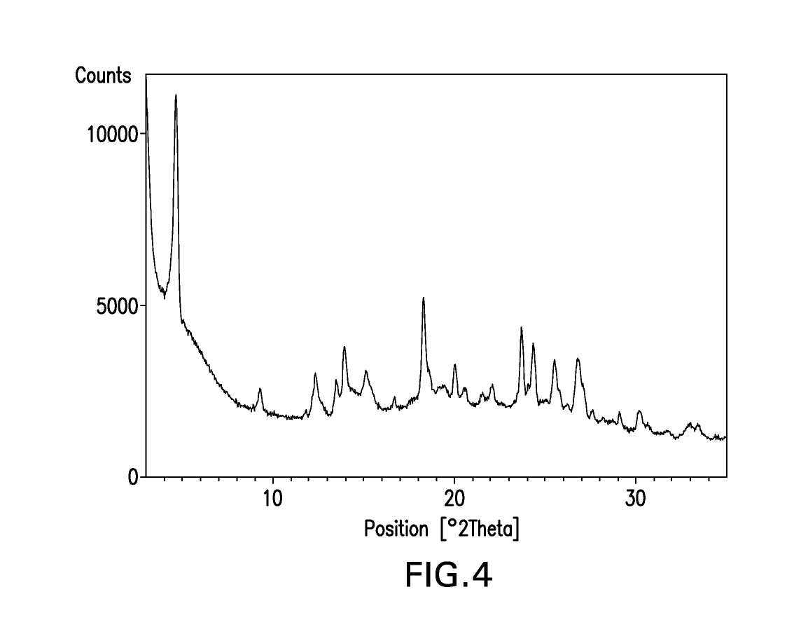

FIG. 4 shows the X-ray diffraction pattern for the crystalline monohydrate form of the bis(sitagliptin) phosphoric acid salt of structural formula IV-a. The monohydrate exhibited diffraction peaks corresponding to d-spacings of 19.0, 4.8, and 3.8 angstroms. The monohydrate was further characterized by the d-spacings of 3.7, 6.4, and 3.3 angstroms. The monohydrate was even further characterized by the d-spacings of 3.5, 4.4, and 5.9 angstroms.

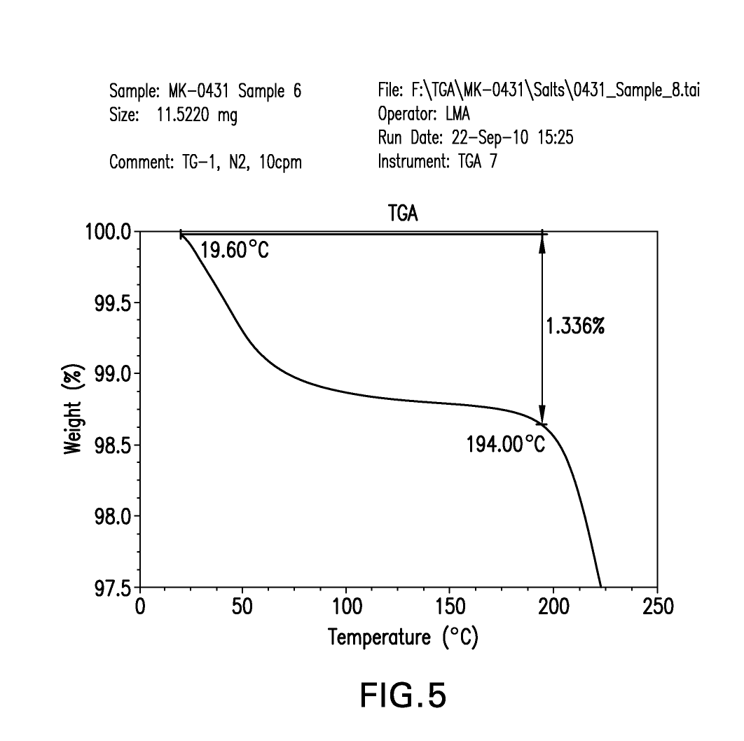

FIG. 5 shows the thermogravimetric analysis (TGA) curve for the crystalline monohydrate form of the bis(sitagliptin) phosphoric acid salt of structural formula IV-a. TG data were acquired using a Perkin Elmer model TGA 7. Experiments were performed under a flow of nitrogen and using a heating rate of 10° C./min to a maximum temperature of approximately 250° C. After automatically taring the balance, 5 to 20 mg of sample was added to the platinum pan, the furnace was raised, and the heating program started. Weight/temperature data were collected automatically by the instrument. Analysis of the results was carried out by selecting the Delta Y function within the instrument software and choosing the temperatures between which the weight loss is to be calculated. Weight losses are reported up to the onset of decomposition/evaporation. TGA indicated a weight loss of about 1.34% from ambient temperature to about 194° C.

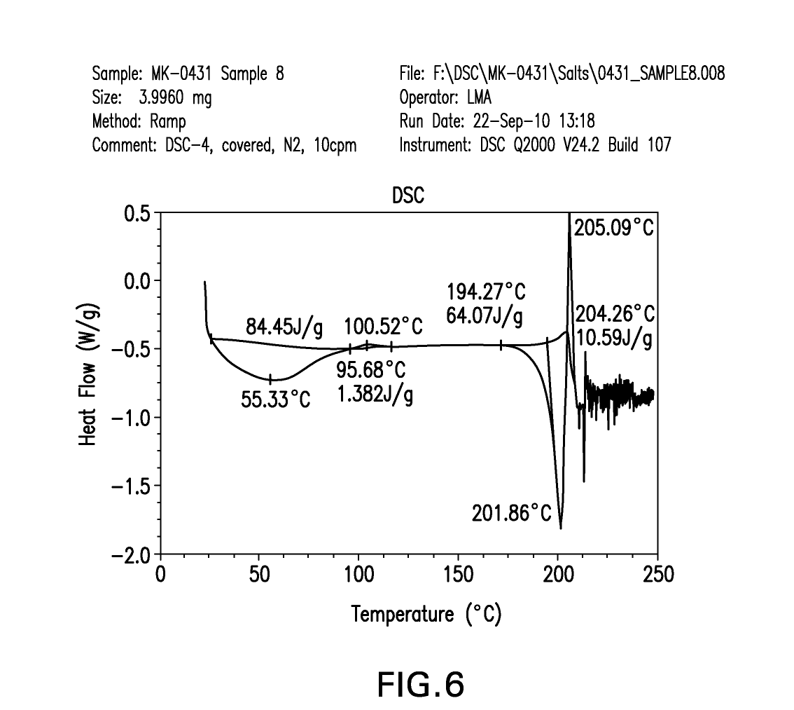

FIG. 6 shows the DSC curve for the crystalline monohydrate form of the bis(sitagliptin) phosphoric acid salt of structural formula IV-a. DSC data were acquired using TA Instruments DSC Q2000 or equivalent. Between 2 and 6 mg sample was weighed into a pan and covered. This pan was then covered and placed at the sample position in the calorimeter cell. An empty pan was placed at the reference position. The calorimeter cell was closed and a flow of nitrogen was passed through the cell. The heating program was set to heat the sample at a heating rate of 10° C./min to a temperature of approximately 250° C. The heating program was started. When the run was completed, the data were analyzed using the DSC analysis program contained in the system software. The thermal events were integrated between baseline temperature points that are above and below the temperature range over which the thermal event was observed. The data reported were the onset temperature, peak temperature and enthalpy.

### Example 3

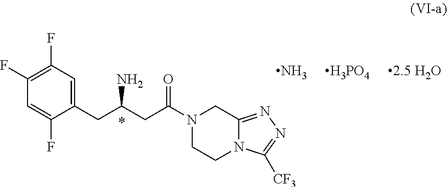

### [(2R)-4-oxo-4-[3-(trifluoromethyl)-5,6-dihydro[1,2,4]triazolo[4,3-a]pyrazin-7(8H)-yl]-1-(2,4A5-trifluorophenyl)butan-2-amine]ammonia phosphoric acid 2.5 hydrate salt (also known as the sitagliptin ammonia phosphoric acid 2.5 hydrate salt)

Sitagliptin free base (1.50 g, 0.00368 moles) was combined with isopropanol (3.2 mL) and distilled water (1.4 mL). The mixture was stirred for 5 to 10 minutes to form a solution. Ammonium phosphate ((NH₄)H₂PO₄, 0.42 g, 0.00365 moles) was added with stirring. The mixture was heated to 70° C. with stirring for 15 minutes and then cooled to room temperature to yield a white crystalline powder. The solid crystalline powder was dried overnight at room temperature under vacuum to give the sitagliptin ammonia phosphoric acid 2.5 hydrate salt, which corresponds to [(sitagliptin) (NH₃) (H₃PO₄) (H₂O)₂.5] salt. Elemental analysis: C (34.36%), H (4.21%), N (14.67%), P (5.50%).

X-ray powder diffraction studies are widely used to characterize molecular structures, crystallinity, and polymorphism. The X-ray powder diffraction pattern of the crystalline sitagliptin ammonia phosphoric acid 2.5 hydrate was generated on a Philips Analytical X'Pert PRO X-ray Diffraction System with PW3040/60 console. A PW3373/00 ceramic Cu LEF X-ray tube K-Alpha radiation was used as the source.

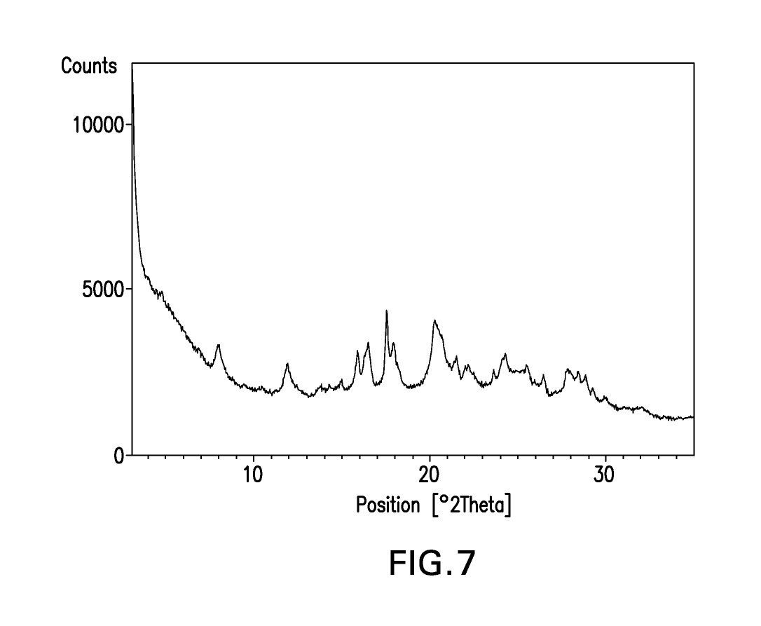

FIG. 7 shows the X-ray diffraction pattern for the crystalline 2.5 hydrate form of the sitagliptin ammonia phosphoric acid salt of structural formula VI-a. The 2.5 hydrate exhibited diffraction peaks corresponding to d-spacings of 5.1, 4.4, and 4.3 angstroms. The 2.5 hydrate was further characterized by the d-spacings of 4.9, 5.4, and 3.7 angstroms. The 2.5 hydrate was even further characterized by the d-spacings of 4.1, 5.6, and 3.5 angstroms.

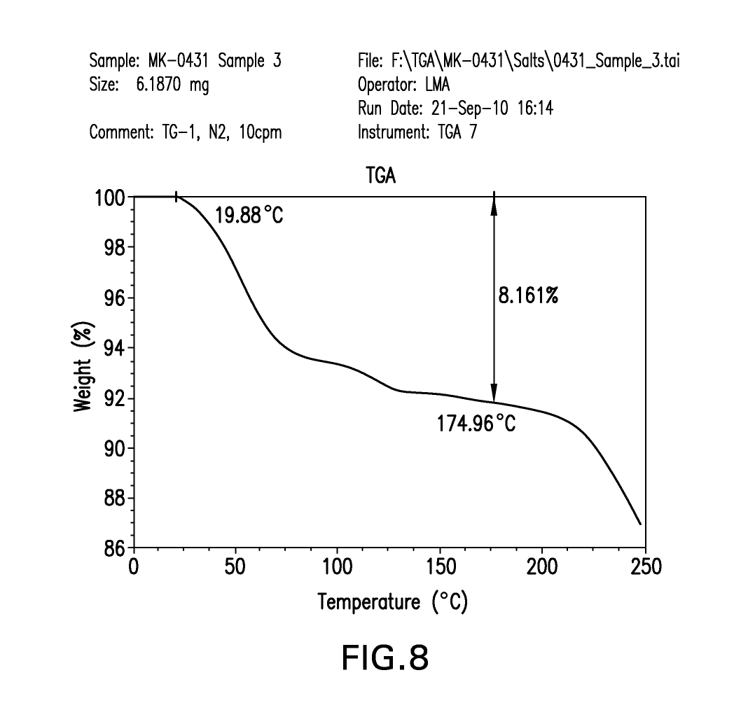

FIG. 8 shows the thermogravimetric analysis (TGA) curve for the crystalline 2.5 hydrate form of the sitagliptin ammonia phosphoric acid salt of structural formula VI-a. TG data were acquired using a Perkin Elmer model TGA 7. Experiments were performed under a flow of nitrogen and using a heating rate of 10° C./min to a maximum temperature of approximately 250° C. After automatically taring the balance, 5 to 20 mg of sample was added to the platinum pan, the furnace was raised, and the heating program started. Weight/temperature data were collected automatically by the instrument. Analysis of the results was carried out by selecting the Delta Y function within the instrument software and choosing the temperatures between which the weight loss is to be calculated. Weight losses are reported up to the onset of decomposition/evaporation. TGA indicated a weight loss of about 8.161% from ambient temperature to about 174.98° C.

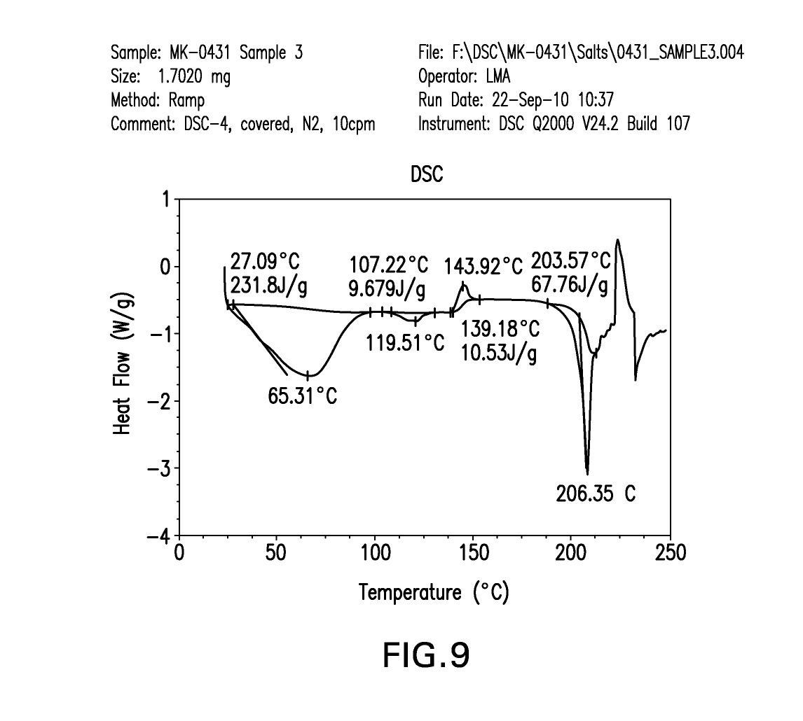

FIG. 9 shows the DSC curve for the crystalline 2.5 hydrate form of the sitagliptin ammonia phosphoric acid salt of structural formula VI-a. DSC data were acquired using TA Instruments DSC Q2000 or equivalent. Between 2 and 6 mg sample was weighed into a pan and covered. This pan was then covered and placed at the sample position in the calorimeter cell. An empty pan was placed at the reference position. The calorimeter cell was closed and a flow of nitrogen was passed through the cell. The heating program was set to heat the sample at a heating rate of 10° C./min to a temperature of approximately 250° C. The heating program was started. When the run was completed, the data were analyzed using the DSC analysis program contained in the system software. The thermal events were integrated between baseline temperature points that are above and below the temperature range over which the thermal event was observed. The data reported were the onset temperature, peak temperature and enthalpy.

### Example 4

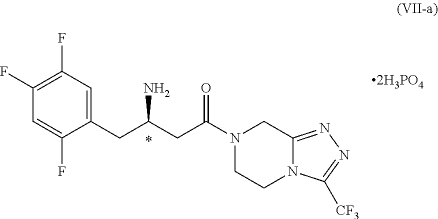 [SMILES: N[C@@H](CC(=O)N1CCn2c(nnc2C(F)(F)F)C1)Cc1cc(F)c(F)cc1F ; "·2H3PO4"]

### [(2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine] bis(phosphoric acid) salt (also known as the sitagliptin bis(phosphoric acid) salt)

Sitagliptin free base (0.75 g, 0.00184 moles) was combined with acetone (4.0 mL). The mixture was stirred for 5 to 10 minutes to form a solution. Anhydrous crystalline H₃PO₄ (0.36 g, 0.00368 moles) was added with stirring. The mixture was heated to 50° C. with stirring for 15 minutes and then cooled to room temperature. The acetone was removed under vacuum at room temperature to yield a white solid. The white solid was crushed with a spatula and then dried under vacuum overnight to give the amorphous sitagliptin bis(phosphoric acid) salt, which corresponds to the [(sitagliptin) (H₃PO₄)₂] salt. Elemental analysis: C (31.80%), H (3.54%), N (10.71%), P (10.10%).

X-ray powder diffraction studies are widely used to characterize molecular structures, crystallinity, and polymorphism. The X-ray powder diffraction pattern of the amorphous sitagliptin bis(phosphoric acid) salt was generated on a Philips Analytical X'Pert PRO X-ray Diffraction System with PW3040/60 console. A PW3373/00 ceramic Cu LEF X-ray tube K-Alpha radiation was used as the source.

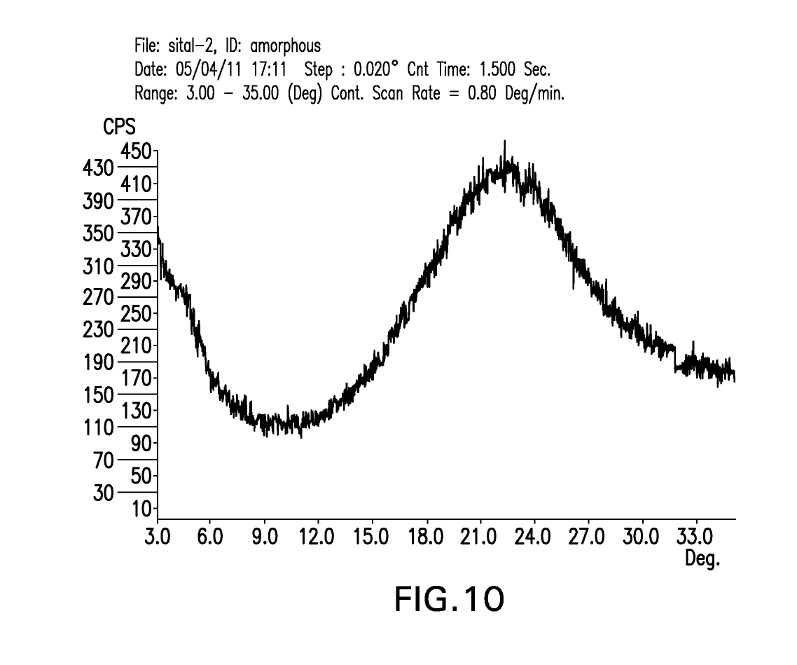

FIG. 10 shows the X-ray diffraction pattern for the amorphous sitagliptin bis(phosphoric acid) salt of structural formula VII-a.

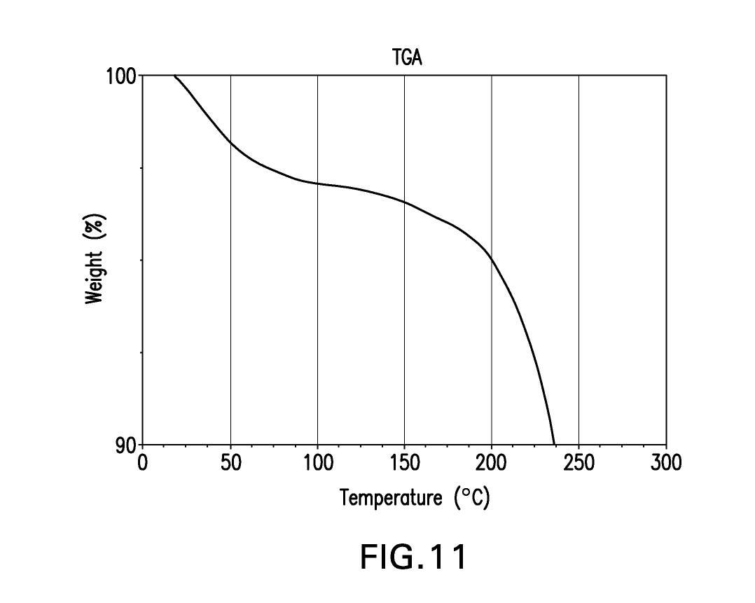

FIG. 11 shows the thermogravimetric analysis (TGA) curve for the amorphous sitagliptin bis(phosphoric acid) salt of structural formula VII-a. TG data were acquired using a Perkin Elmer model TGA 7. Experiments were performed under a flow of nitrogen and using a heating rate of 10° C./min to a maximum temperature of approximately 250° C. After automatically taring the balance, 5 to 20 mg of sample was added to the platinum pan, the furnace was raised, and the heating program started. Weight/temperature data were collected automatically by the instrument. Analysis of the results was carried out by selecting the Delta Y function within the instrument software and choosing the temperatures between which the weight loss is to be calculated. Weight losses are reported up to the onset of decomposition/evaporation.

### Examples of Pharmaceutical Compositions

### 1) Direct Compression Process

The sitagliptin salts of the present invention may be formulated into a tablet by a direct compression process. A 100 mg potency tablet may be composed of 128.4 mg of the active ingredient, 127.8 mg microcrystalline cellulose, 127.8 mg of mannitol (or 127.8 mg of dicalcium phosphate), 8 mg of croscarmellose sodium, 8 mg of magnesium stearate and 16 mg of Opadry white (proprietary coating material made by Colorcon, West Point, Pa.). The active ingredient, microcrystalline cellulose, mannitol (or dicalcium phosphate), and croscarmellose may be blended, and the mixture may then be lubricated with magnesium stearate and pressed into tablets. The tablets may then be film coated with Opadry White.

### 2) Roller Compaction Process

The sitagliptin salts of the present invention may be formulated into a tablet by a roller compaction process. A 100 mg potency tablet may be composed of 128.4 mg of the active ingredient, 45 mg microcrystalline cellulose, 111.6 mg of dicalcium phosphate, 6 mg of croscarmellose sodium, 9 mg of magnesium stearate and 12 mg of Opadry white (proprietary coating material made by Colorcon, West Point, Pa.). The active ingredient, microcrystalline cellulose, dicalcium phosphate, and croscarmellose may be blended, and the mixture may then be lubricated with one third the total amount of magnesium stearate and roller compacted into ribbons. These ribbons may then be milled and the resulting granules may be lubricated with the remaining amount of the magnesium stearate and pressed into tablets. The tablets may then be film coated with Opadry White.

An intravenous (i.v.) aqueous formulation is defined as a sitagliptin salt of the present invention in 10 mM sodium acetate/0.8% saline solution at pH 4.5±0.2. For a formulation with a concentration of 4.0 mg/mL, 800 mg of NaCl is dissolved in 80 mL of water, then 57.5 μL of glacial acetic acid is added, followed by 512 mg of a sitagliptin salt of the present invention. The pH is adjusted to 4.5±0.2 with 0.1N NaOH solution. The final volume is adjusted to 100 mL with water. A 2.0 mg/mL solution can be made by dilution of 50.0 mL of the 4.0 mg/mL solution to 100.0 mL with placebo. A 1.0 mg/mL solution can be made by dilution of 25.0 mL of the 4.0 mg/mL solution to 100.0 mL with placebo.

1. A bis-[4-oxo-4-[3-(trifluoromethyl)-5,6-dihydro[1,2,4]triazolo[4,3-a]pyrazin-7(8H)-yl]-1-(2,4,5-trifluorophenyl)butan-2-amine]phosphoric acid salt of structural formula II: 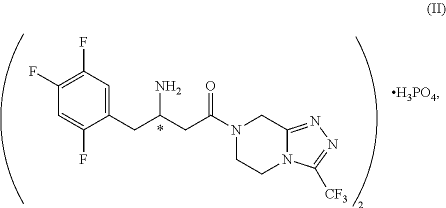
or a polymorph, hydrate and/or solvate thereof.

2. The salt of claim 1 of structural formula II-a having the (R)-configuration at the chiral center marked with an * 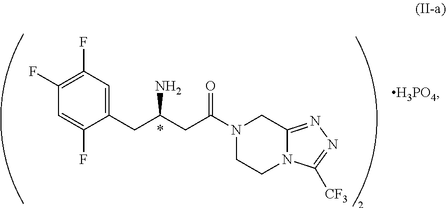
or a polymorph, hydrate and/or solvate thereof.

3. The salt of claim 2 characterized in being a crystalline trihydrate of structural formula III-a:

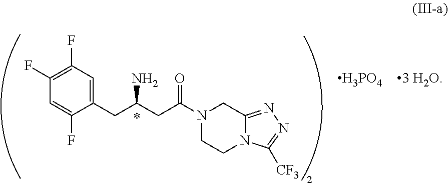

4. The salt of claim 3 characterized by absorption bands obtained from the X-ray powder diffraction pattern at spectral d-spacings of 5.1, 4.0, and 20.1 angstroms.

5. The salt of claim 3 characterized by the thermogravimetric analysis curve of FIG. 2.

6. The salt of claim 3 characterized by the differential scanning calorimetric curve of FIG. 3.

7. The salt of claim 2 characterized in being a crystalline monohydrate of structural formula IV-a:

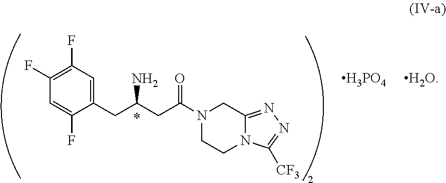

8. The salt of claim 7 characterized by absorption bands obtained from the X-ray powder diffraction pattern at spectral d-spacings of 19.0, 4.8, and 3.8 angstroms.

9. The salt of claim 7 characterized by the thermogravimetric analysis curve of FIG. 5.

10. The salt of claim 7 characterized by the differential scanning calorimetric curve of FIG. 6.

11. A pharmaceutical composition comprising a prophylactically or therapeutically effective amount of the salt according to claim 2, or a pharmaceutically acceptable hydrate thereof, in association with one or more pharmaceutically acceptable carriers.

12. A pharmaceutical composition comprising a prophylactically or therapeutically effective amount of the salt according to claim 3, or a pharmaceutically acceptable solvate thereof, in association with one or more pharmaceutically acceptable carriers.

13. A pharmaceutical composition comprising a prophylactically or therapeutically effective amount of the salt according to claim 7, or a pharmaceutically acceptable solvate thereof, in association with one or more pharmaceutically acceptable carriers.

14. A method for the treatment of type 2 diabetes comprising administering to a patient in need of such treatment a therapeutically effective amount of the salt according to claim 2, or a pharmaceutically acceptable hydrate thereof.

15. A method for the treatment of type 2 diabetes comprising administering to a patient in need of such treatment a therapeutically effective amount of the salt according to claim 3, or a pharmaceutically acceptable solvate thereof.

16. A method for the treatment of type 2 diabetes comprising administering to a patient in need of such treatment a therapeutically effective amount of the salt according to claim 7, or a pharmaceutically acceptable solvate thereof.

17-19. (canceled)

20. A 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine ammonia phosphoric acid salt of structural formula V: 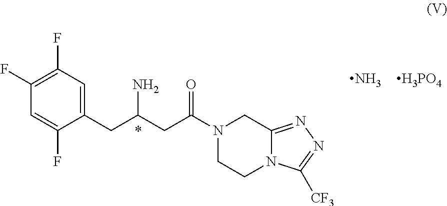
or a polymorph, hydrate and/or solvate thereof.

21. The salt of claim 20 of structural formula V-a having the (R)-configuration at the chiral center marked with an * 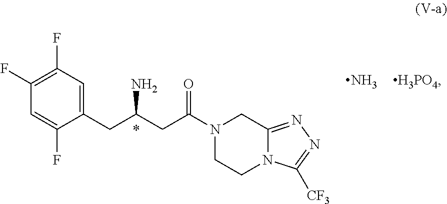
or a polymorph, hydrate and/or solvate thereof.

22. The salt of claim 21 characterized in being a crystalline 2.5 hydrate of structural formula VI-a

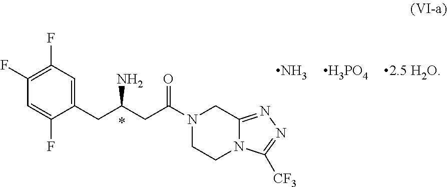

23. The salt of claim 22 characterized by absorption bands obtained from the X-ray powder diffraction pattern at spectral d-spacings of 5.1, 4.4, and 4.3 angstroms.

24. The salt of claim 22 characterized by the thermogravimetric analysis curve of FIG. 8.

25. The salt of claim 22 characterized by the differential scanning calorimetric curve of FIG. 9.

26. A pharmaceutical composition comprising a prophylactically or therapeutically effective amount of the salt according to claim 21, or a pharmaceutically acceptable hydrate thereof, in association with one or more pharmaceutically acceptable carriers.

27. A pharmaceutical composition comprising a prophylactically or therapeutically effective amount of the salt according to claim 22, or a pharmaceutically acceptable solvate thereof, in association with one or more pharmaceutically acceptable carriers.

28. A method for the treatment of type 2 diabetes comprising administering to a patient in need of such treatment a therapeutically effective amount of the salt according to claim 21, or a pharmaceutically acceptable hydrate thereof.

29. A method for the treatment of type 2 diabetes comprising administering to a patient in need of such treatment a therapeutically effective amount of the salt according to claim 22, or a pharmaceutically acceptable solvate thereof.

30-31. (canceled)

32. A 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine bis(phosphoric acid) salt of structural formula VII: 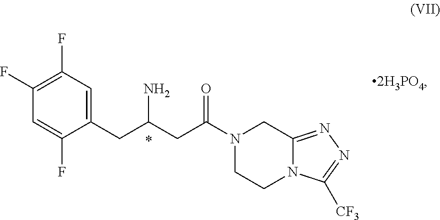 [SMILES: NC(CC(=O)N1CCn2c(nnc2C(F)(F)F)C1)Cc1cc(F)c(F)cc1F]
or a polymorph, hydrate and/or solvate thereof.

33. The salt of claim 32 of structural formula VII-a having the (R)-configuration at the chiral center marked with an * 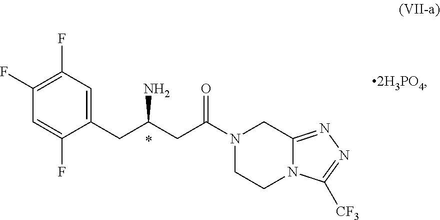 [SMILES: N[C@@H](CC(=O)N1CCn2c(nnc2C(F)(F)F)C1)Cc1cc(F)c(F)cc1F]
or a polymorph, hydrate and/or solvate thereof.

34. The salt of claim 33 characterized by absorption bands obtained from the X-ray powder diffraction pattern of FIG. 10.

35. The salt of claim 33 characterized by the thermogravimetric analysis curve of FIG. 11.

36. A pharmaceutical composition comprising a prophylactically or therapeutically effective amount of the salt according to claim 32, or a pharmaceutically acceptable hydrate thereof, in association with one or more pharmaceutically acceptable carriers.

37. A pharmaceutical composition comprising a prophylactically or therapeutically effective amount of the salt according to claim 33, or a pharmaceutically acceptable solvate thereof, in association with one or more pharmaceutically acceptable carriers.

38. A method for the treatment of type 2 diabetes comprising administering to a patient in need of such treatment a therapeutically effective amount of the salt according to claim 32, or a pharmaceutically acceptable hydrate thereof.

39. A method for the treatment of type 2 diabetes comprising administering to a patient in need of such treatment a therapeutically effective amount of the salt according to claim 33, or a pharmaceutically acceptable solvate thereof.

40-41. (canceled)
